(19) United States
(12) Patent Application Publication
(10) Pub. No.: US 2013/0123885 A1
(43) Pub. Date: May 16, 2013

(54) APPARATUS, SYSTEMS, COMPUTER-ACCESSIBLE MEDIUM AND METHODS FOR FACILITATING RADIO FREQUENCY HYPERTHERMIA AND THERMAL CONTRAST IN A MAGNETIC RESONANCE IMAGING SYSTEM

(71) Applicants: Yudong Zhu, Scarsdale, NY (US); Leeor Alon, New York, NY (US)

(72) Inventors: Yudong Zhu, Scarsdale, NY (US); Leeor Alon, New York, NY (US)

(73) Assignee: NEW YORK UNIVERSITY, New York, NY (US)

(21) Appl. No.: 13/641,641

(22) PCT Filed: Apr. 15, 2011
(86) PCT No.: PCT/US11/32747

§ 371 Date: Jan. 31, 2013

Related U.S. Application Data
(60) Provisional application No. 61/325,130, filed on Apr. 16, 2010.

(51) Int. Cl. A61N 5/00

(52) U.S. Cl. CPC A61N 5/00
USPC 607/100

(57) ABSTRACT

The present disclosure can provide exemplary apparatus, system, methods, and computer-accessible medium for generating a prediction model for at least one of an electromagnetic radiation absorption or a specific absorption rate (SAR). For example, according to certain exemplary embodiments, an exemplary method can include directing electromagnetic radiation at a subject using a plurality of transmit elements, obtaining electromagnetic radiation deposition information associated with a deposition of the at least one electromagnetic radiation within the subject using a sensing apparatus, and with a processor arrangement, generating the prediction model as a function of the electromagnetic radiation deposition information.

### CROSS-REFERENCE TO PRIOR APPLICATIONS

This application claims priority to U.S. Provisional Application Ser. No. 61/325,130, filed on Apr. 16, 2010, which is incorporated by reference herein in its entirety.

### FIELD OF THE DISCLOSURE

Exemplary embodiments of the present disclosure relate generally to apparatus, systems, computer-accessible medium and methods for facilitating treatment using a magnetic resonance imaging (MRI) system, and more particularly to exemplary embodiments of apparatus, systems, computer-accessible medium and methods for facilitating radio frequency hyperthermia and thermal contrast in the MRI system.

### BACKGROUND INFORMATION

In cancer treatment, hyperthermia has been shown to be an effective method for treating cancer malignancies, especially when used in conjunction with chemotherapy or radiotherapy. Radio frequency (RF) hyperthermia, in particular, can be used to create spatially localized heating patterns within a subject body, providing a treatment option that can be noninvasive and have relatively minimum side effects when compared to other treatment options. Conventional RF hyperthermia can use an array antenna surrounding the body and deliver RF energy to a target region by controlling the amplitudes and/or phases of RF waveforms driving each individual element in the array antenna. Monitoring of the localized heating pattern can be performed using thermocouples that directly measure the temperature rise and/or using non-invasive magnetic resonance (MR) and/or infrared thermometry.

EM field numerical simulations can be used to, e.g., evaluate interactions of EM fields with body and provide temporal and spatial information about the internal variation of electric fields, magnetic fields, currents and energy deposition. Although these numerical simulations can often be useful, a weakness of these simulations can be the accuracy of numerical representations in the simulations in comparison with “real life” conditions. This concern is mostly related to the fact that the anatomy of the imaged individual can differ (quite substantially in some instances) from the anatomy of a human body's numerical representation in simulation and that certain assumptions regarding the apparatus can be subject to errors. This discrepancy between electro-magnetic (EM) numerical simulations and the true distributions of internal electric fields, magnetic fields, currents and, likely most importantly, energy deposition, can be a concern for relying on simulations to ensure safe operation of MRI or to plan the hyperthermia treatment for each individual.

### Cancer Related Hyperthermia

Hyperthermia can be a thermal modality that can have significant potential in treating cancer. (See, e.g., Hand, J. W., Modelling the interaction of electromagnetic fields (10 MHz-10 GHz) with the human body: methods and applications, Phys Med Biol, 2008. 53(16): pp. R243-86; Jones, E. L., et al., Randomized trial of hyperthermia and radiation for superficial tumors, J Clin Oncol, 2005. 23(13): pp. 3079-85; and van der Zee, J., et al., Comparison of radiotherapy alone with radiotherapy plus hyperthermia in locally advanced pelvic tumours: a prospective, randomised, multicentre trial, Dutch Deep Hyperthermia Group. Lancet, 2000. 355(9210): pp. 1119-25). Hyperthermia typically raises the temperature of a target tissue to therapeutic levels, for example between approximately 42 degrees centigrade and approximately 45 degrees centigrade, while keeping functional-normal tissue at lower temperatures. This type of treatment is often utilized in conjunction with radiotherapy and/or chemotherapy to improve the life expectancy of cancer patients. Frequently, inherent acidity and/or low oxygen tension in tumors can provide resistance to chemotherapy and/or radiation, although the same factors can render cells more susceptible to heat therapy. Additionally, cytotoxicity of several antineoplastic drugs can be enhanced at higher temperatures (see, e.g., Hand, J. W., Modelling the interaction of electromagnetic fields (10 MHz-10 GHz) with the human body: methods and applications, Phys Med Biol, 2008. 53(16): pp. R243-86; Iliakis, G., et al., Evidence for an S-phase checkpoint regulating DNA replication after heat shock: a review, Int J Hyperthermia, 2004. 20(2): pp. 240-9; Mahaley, M. S., Jr., B. Woodhall, and W. H. Knisely, Selection of anti-cancer agents for regional brain cancer perfusion. Surg Forum, 1960. 10: pp. 774-7; and Takemoto, M., et al., The effect of various chemotherapeutic agents given with mild hyperthermia on different types of tumours, Int J Hyperthermia, 2003. 19(2): pp. 193-203) and therefore improving the effects of some chemotheraputics. For example, hyperthermia added to current treatment regiments have reported as much as a 50% improvement in response rates, tumor control rates, and overall survival (see, e.g., van der Zee, J., Heating the patient: a promising approach? Ann Oncol, 2002. 13(8): pp. 1173-84), even though, “the task of delivering hyperthermia therapy in a controlled and predictable manner has proven to be challenging.” (See, e.g., Hand, J. W., Modelling the interaction of electromagnetic fields (10 MHz-10 GHz) with the human body: methods and applications, Phys Med Biol, 2008. 53(16): pp. R243-86).

Since the delivery and control of a correct, optimal and/or preferred thermal dose has been a difficult task, it is believed that many patients may have received an incorrect (e.g., suboptimal) amount of thermal dose. This incorrect application of thermal dose can be due to variations in anatomy, position of the body relative to the applicator and more. Additionally, numerical simulation studies that have been used to decide the thermal dosage have not been sufficiently accurate. In fact, on the difficulty of applying an accurate heating dosage, several studies, for example, have indicated the effect that under modest temperature increase, tumor oxygenation can significantly increase causing adverse effects such as tumor proliferation. (See, e.g., Tumour oxygenation is increased by hyperthermia at mild temperatures. 2009. 25(2): pp. 95). Further, Bicher et al., Effects of hyperthermia on normal and tumor microenvironment, Radiology, 1980. 137(2): pp. 523-30; and Bicher, H. I., The physiological effects of hyperthermia, Radiology, 1980. 137(2): pp. 511-513, for example, indicated that the pO₂ in a C3H mouse mammary adenocarcinoma increased as a result of an increase in tumor blood flow when the subject tumors were heated at temperatures below 41 degrees centigrade, and decreased as the heating temperature was raised to over 41 degrees centigrade. Vaupel et al. (see, e.g., Vaupel, P., et al., Oxygenation of mammary tumors: from isotransplanted rodent tumors to primary malignancies in patients, Adv Exp Med Biol, 1992. 316: pp. 361-71) reported, for example, that oxygenation in DS-carcinoma in rats can be significantly improved upon heating at about 40 degrees centigrade, and decline when heating is raised to about 43 degrees centigrade. These and other results may indicate and/or emphasize that prescribing and controlling spatially and temporally the correct (e.g., optimal, preferred, etc.) dose of thermal therapy can be important. E. Jones et al (see, e.g., Jones, E., et al., Prospective thermal dosimetry: the key to hyperthermia's future, Int J Hyperthermia, 2006. 22(3): pp. 247-53) describes, for example, that efforts to refine three dimensional thermal dose distributions with non-invasive MR-based thermometry may enhance the progress that has been made with regard to characterizing thermal dose and that ultimately, these efforts will optimize the clinical applications of hyperthermia. (See, e.g., Jones, E., et al., Prospective thermal dosimetry: the key to hyperthermia's future, Int J Hyperthermia, 2006. 22(3): pp. 247-53). This prediction capability can be a strength of the exemplary local power deposition prediction model in accordance with certain exemplary embodiments of the present disclosure. Heretofore, numerical simulations and relatively simple phantom studies have been used to map power deposition. (See, e.g., B, B. B. and et al., Comparisons of computed mobile phone induced SAR in the SAM phantom to that in anatomically correct models of the human head, 2006. 48(2): pp. 397; Christ A, C. N., Nikoloski N, Gerber H, Pokovic K and K. N, A numerical and experimental comparison of human head phantoms for compliance testing of mobile telephone equipment, 2005. 26(2): pp. 125; de Bree, J., J. F. van der Koijk, and J. J. Lagendijk, A 3-D SAR model for current source interstitial hyperthermia, IEEE Trans Biomed Eng, 1996. 43(10): pp. 1038-45; Dimbylow, P. J. and S. M. Mann, SAR calculations in an anatomically realistic model of the head for mobile communication transceivers at 900 MHz and 1.8 GHz. Phys Med Biol, 1994. 39(10): pp. 1537-53; Gandhi O P, L. G. and F. C. M, Electromagnetic absorption in the human head and neck for mobile telephones at 835 and 1900 MHz. 1996. 44(10): pp. 1884; Hand J W, v.L.G.M.J., Mizushina S, Van de Kamer J B, Maruyama K, Sugiura T, Azzopardi D V and E. A. D, Monitoring deep brain temperature in infants using multifrequency microwave radiometry and thermal modelling. 2001. 46(7): pp. 1885; Hombach V, M. K., Burkhardt M, Kuhn E and K. N, The dependence of EM energy absorption upon human head modeling at 900 MHz. 1996. 44(10): pp. 1865; IEC, 60601-2-33 Medical Electrical Equipment: Part 2. Particular requirements for the Safety of Magnetic Resonance Equipment for Medical Diagnosis. 2002; IEC, 62209-1 Human exposure to radio frequency fields from hand-held and bodymounted wireless communication devices-Human models, instrumentation, and procedures: Part 1. Procedure to determine the specific absorption rate (SAR) for handheld devices used in close proximity to the ear (frequency range of 300 MHz to 3 GHz) 2005; Keshvari, J., R. Keshvari, and S. Lang, The effect of increase in dielectric values on specific absorption rate (SAR) in eye and head tissues following 900, 1800 and 2450 MHz radio frequency (RF) exposure. Phys Med Biol, 2006. 51(6): pp. 1463-77). these studies apparently were not subject specific and held no predictive capabilities of the fields.

Hyperthermia for Treatment of Articular Cartilage with Osteoarthritis

Osteoarthritis (OA) is a most frequent musculoskeletal disorders in the elderly population. The process of OA, which likely has a genetic basis, can be accelerated by injury and/or disease. For example, OA affects approximately 60% of men and 70% of women after the age of 65 years, and in the United States it affects about 27 million people. Additionally, current medical therapies may not be available or able to delay the onset of joint degradation. (See, e.g., Ulrich-Vinther, M., et al., Articular cartilage biology. J Am Acad Orthop Surg, 2003. 11(6): pp. 421-30). OA can be characterized by a gradual loss of extracellular matrix in the articular cartilage of joints. For example, since the joint function can be severely impaired, personal and/or social activities of patients with OA can be limited. (See, e.g., Takahashi, K. A., et al., Hyperthermia for the treatment of articular cartilage with osteoarthritis. Int J Hyperthermia, 2009. 25(8): pp. 661-7). Because articular cartilage does not have blood vessels, it is possible that the cells that repair the damaged cartilage cannot access the damaged area. Chondrocytes themselves can have limitations in their proliferative potential and/or repair capacity, therefore, when OA progresses and the articular cartilage degenerates, it can become difficult to treat. Generally, heat therapy has been used to treat OA successfully by, for example, improving the decreasing muscle spasms, increasing collagen extensibility and accelerating metabolic processes. For example, Tonomura et al. (see, e.g., Tonomura, H., et al., Effects of heat stimulation via microwave applicator on cartilage matrix gene and HSP70 expression in the rabbit knee joint. J Orthop Res, 2008. 26(1): pp. 34-41) indicated that applied heat stimulation to the knee joints of rabbits for 20 minutes using a clinical available 2.45-GHz applicator can produce desirable results. In this work, the Rabbits' joints were heated up to approximated 40 degrees centigrade and increased expression of proteoglycan and type II collagen in the articular cartilage was observed. Additionally, heat shock protein 70 (HSP70), which is a protein that can have a protective effect on the cartilage and inhibit the apoptosis of chondrocytes, has been observed to accumulate in the chondrocytes.

Conventional ultrasound treatment can include a method of treating OA, however, the precise control and monitoring of the thermal dosage has not been done in OA treatment.

### Enhanced Temperature-Based Drug Delivery and Contrast

There have been studies that explore the use of hyperthermia as a way to initiate a temperature-dependent release of drug. For example, in particles such as certain liposomes, increased localized temperatures can create an enhancement in the permeability of the encapsulating membrane and trigger a rapid localized release of the drug. For example, anti-angiogenic drugs can create a “normalization” window in the tumor microvasculature, during which delivery of a chemotherapeutic drug can be most effective in the treatment against cancer. (See, e.g., Van der Zee, J., et al., Comparison of radiotherapy alone with radiotherapy plus hyperthermia in locally advanced pelvic tumours: a prospective, randomised, multicentre trial. Dutch Deep Hyperthermia Group. Lancet, 2000. 355(9210): pp. 1119-25; Iliakis, G., et al., Evidence for an S-phase checkpoint regulating DNA replication after heat shock: a review. Int J Hyperthermia, 2004. 20(2): pp. 240-9; Bicher, H. I., The physiological effects of hyperthermia. Radiology, 1980. 137(2): pp. 511-3; and Vaupel, P., et al., Oxygenation of mammary tumors: from isotransplanted rodent tumors to primary malignancies in patients. Adv Exp Med Biol, 1992. 316: pp. 361-71).

Additionally, with advancements in the detection and/or treatment of cancer, it is believed that the survival rate of cancer has generally significantly improved. However, even as research develops more specific and efficient drug treatments against cancer, there can still be much that is unknown about the heterogeneous tumor microenvironment of cancer, preventing effective drug delivery. (See, e.g., Hanahan, D. and R. A. Weinberg, The Hallmarks of Cancer. Cell, 2000. 100(1): pp. 57-70). Therefore, providing an exemplary accurate non-invasive measurement and/or analysis of the tumor microenvironment can significantly improve the efficacy of drug timing and/or delivery as well as providing additional information with respect to the properties of the tumor microenvironment including, e.g., perfusion changes in the tumor microvasculature and high interstitial fluid pressure (IFP). (See, e.g., Baxter, L. T. and R. K. Jain, Transport of fluid and macromolecules in tumors. I. Role of interstitial pressure and convection. Microvascular Research, 1989. 37(1): pp. 77-104; Fukumura, D. and R. K. Jain, Tumor microvasculature and microenvironment: Targets for anti-angiogenesis and normalization. Microvascular Research. 74(2-3): pp. 72-84; Fukumura, D. and R. K, Jain, Tumor microenvironment abnormalities: Causes, consequences, and strategies to normalize. Journal of Cellular Biochemistry, 2007. 101(4): pp. 937-949; FUKUMURA, D. and R. K. JAIN, Imaging angiogenesis and the microenvironment&nbsp. APMIS, 2008. 116(7-8): pp. 695-715). It is believed that high IFP, which can be considered to be a well known hallmark of cancer (see, e.g., Gullino, P. M., S. H. Clark, and F. H. Grantham, The Interstitial Fluid of Solid Tumors. Cancer Res, 1964. 24(5): pp. 780-797) and complex branching, increased vessel diameter and permeability, and/or reduced microvascular flow can be hallmarks potentially quantifiable by MR imaging. (See, e.g., Baxter, L. T. and R. K. Jain, Transport of fluid and macromolecules in tumors. I. Role of interstitial pressure and convection. Microvascular Research, 1989. 37(1): pp. 77-104; Fukumura, D. and R. K. Jain, Tumor microvasculature and microenvironment: Targets for anti-angiogenesis and normalization. Microvascular Research. 74(2-3): pp. 72-84; Fukumura, D. and R. K. Jain, Tumor microenvironment abnormalities: Causes, consequences, and strategies to normalize. Journal of Cellular Biochemistry, 2007. 101(4): pp. 937-949; FUKUMURA, D. and R. K. JAIN, Imaging angiogenesis and the microenvironment&nbsp. APMIS, 2008. 116(7-8): pp. 695-715; Bicher, H. I., et al., Effects of hyperthermia on normal and tumor microenvironment. Radiology, 1980. 137(2): pp. 523-30; and Bicher, H. I., The physiological effects of hyperthermia. Radiology, 1980. 137(2): pp. 511-3). It is possible for rapid IFP buildup in tumors for both human and animal cases to be created by the formation of abnormal and/or highly permeable tumor blood vessels. (See, e.g., Fukumura, D. and R. K. Jain, Tumor microvasculature and microenvironment: Targets for anti-angiogenesis and normalization. Microvascular Research. 74(2-3): pp. 72-84; Fukumura, D. and R. K. Jain, Tumor microenvironment abnormalities: Causes, consequences, and strategies to normalize. Journal of Cellular Biochemistry, 2007. 101(4): pp. 937-949; Leunig, M., et al., Angiogenesis, Microvascular Architecture, Microhemodynamics, and Interstitial Fluid Pressure during Early, Growth of Human Adenocarcinoma LS174T in SCID Mice. Cancer Res, 1992. 52(23): pp. 6553-6560; and Carmeliet, pp. and R. K. Jain, Angiogenesis in cancer and other diseases. Nature, 2000. 407(6801): pp. 249-257). Permeability can facilitate/provide for a large amount of fluid to transfer into the interstitial space, which can lead to a rise in pressure that can compress the lymphatic thereby likely preventing drainage and increasing pressure even further. (See, e.g., Baxter, L. T. and R. K. Jain, Transport of fluid and macromolecules in tumors. I. Role of interstitial pressure and convection. Microvascular Research, 1989. 37(1): pp. 77-104; and Boucher, Y., M. Leunig, and R. K. Jain, Tumor Angiogenesis and Interstitial Hypertension. Cancer Res, 1996. 56(18): pp. 4264-4266). Eventually, the pressure in the intersitium can equal the pressure in the vessels, which can create a physiological barrier of high resistance to flow, for example. This barrier can prevent the delivery of chemotherapeutics into the system, and can possibly be altered by applying hyperthermia therapy. (See, e.g., Leunig, M., et al., Angiogenesis, Microvascular Architecture, Microhemodynamics, and Interstitial Fluid Pressure during Early Growth of Human Adenocarcinoma LS174T in SCID Mice. Cancer Res, 1992. 52(23): pp. 6553-6560; Jain, R. K., Vascular and interstitial barriers to delivery of therapeutic agents in tumors. Cancer and Metastasis Reviews, 1990. 9(3): pp. 253-266; and Jain, R. K., A. W. Cook, and E. L. Steele, Haemodynamic and Transport Barriers to the Treatment of Solid Tumours. International Journal of Radiation Biology, 1991. 60(1-2): pp. 85-100). It is possible that this can manifest in a thermal response-based tissue contrast.

### SUMMARY OF EXEMPLARY EMBODIMENTS OF THE DISCLOSURE

Indeed, certain exemplary embodiments of the present disclosure can address the exemplary problems described herein above, and/or overcome the exemplary deficiencies commonly associated with the prior art as, e.g., described herein.

As described herein, among potential applications of certain exemplary embodiments according to the present disclosure can facilitate an exemplary thermal response-based contrast for identifying cancer and other tissue abnormalities. This exemplary diagnostic method, apparatus, etc. can facilitate monitoring of the progression of cancer and effects of cancer treatment by better understanding of tumor micro-vascular environment and its response to heating.

Exemplary embodiments according to the present disclosure can provide an exemplary RF hyperthermia apparatus, systems and methods in which an exemplary parallel RF transmit-capable MR scanner can be used to perform RF hyperthermia. In addition, exemplary embodiments of a method and/or procedure according to the present disclosure can be provided, which can be utilized to improve treatment planning and/or execution by leveraging an exemplary noninvasive approach to RF energy deposition pattern prediction. According to additional exemplary embodiments of the present disclosure, optimized hyperthermia, as described herein, can potentially improve localization of drug delivery, for example.

The exemplary integration of RF hyperthermia and MR imaging in accordance with certain exemplary embodiments of the present disclosure can facilitate MRI-based thermal dose calibration, target thermal dose delivery, real-time monitoring and evaluation of the therapeutic effects, and/or continuous adaptation and/or feedback control of RF waveforms for precise thermal dose delivery, for example. This exemplary integration can also provide potential benefits to overall cost. According to further exemplary embodiments of the present disclosure, apparatus, systems and methods can be provided for facilitating a noninvasive approach to RF energy deposition pattern prediction, including treatment planning and execution.

For example, using an exemplary mathematical formulation, (see, e.g., Zhu, Y., Parallel excitation with an array of transmit coils. Magn Reson Med, 2004. 51(4): pp. 775-84) field maps of power deposition can be mapped and predicted. Indeed, according to still another exemplary embodiment of the present disclosure, subject specific exemplary method and/or process can be provided for, e.g., measuring and/or predicting power deposition for RF hyperthermia therapy. For example, once the local RF power deposition model is calibrated, the model can possess the information that can be needed to predict the local power deposition maps for any array coil, pulse amplitude and phase. This exemplary prediction capability can be applied to, e.g., monitoring MR based hyperthermia therapy and/or optimization of RF pulses such that a region of interest (ROI) can be heated to therapeutic levels more precisely and controllably while normal tissue does not reach such temperatures.

For example, described herein is an exemplary procedure according to certain exemplary embodiments of the present disclosure for, e.g., controlling a deposition of an electromagnetic energy in a region of interest (ROI) of a subject using a plurality of transmit elements. The exemplary procedure can include, e.g., obtaining first information associated with a calibration of the transmit elements relative to the subject, determining second information associated with a temporal change of at least one of a phase or an amplitude waveform configured to drive the transmit elements utilizing the first information, and controlling at least one of the phase or the amplitude waveform of at least one of the transmit elements utilizing the second information. The first information can be determined as a function of at least one of a constructive interference of the transmit elements or a destructive interference of the transmit elements, and can include further information associated with a relationship between the transmit elements and the subject.

The exemplary procedure can further include facilitating the delivery of a predetermined dose of the electromagnetic energy using the first information and/or the second information. Further, according to certain exemplary embodiments of the present disclosure, the exemplary procedure can further include determining further information associated with a relationship of a normal response of biologic tissue to the electromagnetic radiation and an abnormal response to the electromagnetic energy, where the further information can be based on a pathology of abnormal tissue. It is also possible to determine, using the further information, a contrast configured to be used for diagnostic purposes. The exemplary procedure can also include a control of the deposition of electromagnetic energy inside the ROI, based on the first information and/or the second information.

According to certain exemplary embodiments of the present disclosure, the exemplary procedure can further include facilitating the delivery of a drug to the ROI, where a release of the drug is based on the dose of the electromagnetic radiation, for example. The exemplary procedure can further include receiving temperature data, and, using the temperature data and the first information and/or the second information, determining an actual energy deposition relative to an expected energy deposition. The temperature data can be received from an MR thermometry source and/or an infrared source. Further, the exemplary procedure can include an analysis of a spatial distribution of the energy deposition in the ROI for an element when the element is driven by the waveform. The exemplary plurality of transmit elements can include only elements that deposit an energy greater than a predetermined threshold within the ROI in accordance with certain exemplary embodiments of the present disclosure. It is also possible for the calibration to be subject-specific.

The exemplary procedure can further include determining a local power deposition of a voxel in the ROI as a function of a relationship of electric fields induced by the transmit elements, using the first information and/or the second information. It is possible to determine a local power deposition of a voxel in the ROI as a function of a local power deposition model, for example.

According to additional exemplary embodiments of the present disclosure, an MRI system can be utilized for the MR imaging procedure and/or the controlling procedure. Further, the controlling procedure can be based on providing a hyperthermia treatment, which treatment can be configured for treating a tumor, arthritis and/or a further treatment that can involve deep tissue heating.

Another exemplary embodiment of a method for, e.g., facilitating magnetic resonance imaging can be provided, which can include a determination of a local power deposition model, inducing heat inside tissue in a region of interest, subtracting a measured heat induced from a predicted heat associated with a plurality of voxels, and determining a map associated with at least one of a perfusion or a diffusion as a function of the subtraction. The exemplary method can further include certain procedures, including but not limited to obtaining the measured heat induced from a magnetic resonance thermometry, obtaining the measured heat induced using a diffusion weighted imaging procedure, and/or obtaining the measured heat induced using a proton resonance frequency shift. This exemplary embodiment can provide a contrast related to the response to heating of tissues.

Exemplary embodiments of computer-accessible medium and systems for facilitating the exemplary procedures described herein above are also described herein, for example.

Exemplary embodiments of the present disclosure can provide a method for obtaining information associated with a deposition of at least one electromagnetic radiation in a subject using a plurality of transmit elements. For example, the exemplary method can include directing electromagnetic radiation at or to the subject using the transmit elements, and with a processor arrangement, obtaining the information within the subject using a magnetic resonance (MR) scanning apparatus. The electromagnetic radiation can include a radio frequency (RF) energy, and the directing procedure can include controlling at least one of a phase or a magnitude associated with a current of at least one of the transmit elements. Further, the information can be determined as a function of at least one of a constructive interference of the transmit elements or a destructive interference of the transmit elements, and the information can include further information associated with a relationship between the transmit elements and the subject.

According to yet another exemplary embodiment of the present disclosure, a method for generating a prediction model for at least one of an electromagnetic radiation absorption or a specific absorption rate (SAR) can be provided. The exemplary method can include directing at least one electromagnetic radiation at a subject using a plurality of transmit elements, obtaining electromagnetic radiation deposition information associated with a deposition of the electromagnetic radiation within the subject using a sensing apparatus, and with a processor arrangement, generating the prediction model as a function of the electromagnetic radiation deposition information. The electromagnetic radiation can include a radio frequency (RF) energy. Further, directing procedure can include controlling at least one of a phase or a magnitude associated with a current of at least one of the transmit elements and the generating procedure can include converting the electromagnetic radiation deposition information and the phase or magnitude to the prediction model. According to certain exemplary embodiments, t the electromagnetic radiation deposition information comprises actual temperature data, which can be obtained using at least one of a magnetic resonance thermometry source, an infrared source, or an embedded temperature sensing source.

Certain exemplary embodiments of the present disclosure can include facilitating a delivery of a predetermined dose of the electromagnetic radiation to the subject based on the prediction model and predicting a local electromagnetic radiation power deposition as a function of at least one driving waveform and the prediction model. The driving waveform can be configured to shape a spatial distribution of the local electromagnetic radiation power deposition, and the spatial distribution of the local electromagnetic radiation power deposition can be configured to avoid a substantial concentration of electromagnetic radiation power deposition. Further, the spatial distribution of the local electromagnetic radiation power deposition can be configured to target a pre-defined electromagnetic radiation power deposition profile.

Further exemplary embodiments of the present disclosure can provide controlling at least one of a phase or an amplitude of waveforms of at least one of the transmit elements as a function of the prediction model so as to perform an electromagnetic radiation deposition, which can include monitoring a progress of the electromagnetic radiation energy deposition. The monitoring procedure can include implementing at least one of magnetic resonance imaging or thermometry. Further, the controlling procedure can facilitate the electromagnetic radiation energy deposition and provides a hyperthermia treatment, which can be configured to treat at least one of a tumor, arthritis or deep tissue heating.

Certain further exemplary embodiments of the present disclosure can provide determining further information associated with a relationship of a normal response of biologic tissue to an electromagnetic radiation to be provided to the subject and an abnormal response to the electromagnetic radiation, where the further information can be based on a pathology of an abnormal tissue; and determining, using the further information, a contrast configured to be used for diagnostic purposes.

Certain further exemplary embodiments of the present disclosure can provide facilitating a delivery and release of a drug to the subject, where the release of the drug can be based on a spatially discriminating dose of the electromagnetic radiation to be provided to the subject.

Yet another exemplary embodiment of the present disclosure can provide a method for facilitating magnetic resonance imaging. The method can include determining a local RF power deposition model associated with a subject, inducing heat inside tissue in a region of interest of the subject, using a computing arrangement, subtracting a measured heat induced from a predicted heat associated with a plurality of voxels, and determining a map associated with at least one of a perfusion or a diffusion of the region of interest as a function of the subtraction. The exemplary method can further include obtaining the measured heat induced from a magnetic resonance thermometry associated with the subject and obtaining the measured heat induced using a diffusion weighted imaging procedure associated with the subject. The method can further include obtaining the measured heat induced using a proton resonance frequency shift associated with the subject.

Yet another exemplary embodiment of the present disclosure can provide a computer-accessible medium having instructions thereon for generating a prediction model for at least one of an electromagnetic radiation absorption or a specific absorption rate (SAR). When a hardware processing arrangement executes the instructions, the hardware arrangement can be configured to direct at least one electromagnetic radiation at a subject using a plurality of transmit elements, obtain electromagnetic radiation deposition information associated with a deposition of the electromagnetic radiation within the subject using a sensing apparatus, and generate the prediction model as a function of the electromagnetic radiation deposition information.

Yet another exemplary embodiment of the present disclosure can provide a system for generating a prediction model for at least one of an electromagnetic radiation absorption or a specific absorption rate (SAR). The system can include a computer-accessible medium having executable instructions thereon, and when at least one hardware processing arrangement executes the instructions, the hardware processing arrangement can be configured to direct at least one electromagnetic radiation at a subject using a plurality of transmit elements, obtain electromagnetic radiation deposition information associated with a deposition of the electromagnetic radiation within the subject using a sensing apparatus, and generate the prediction model as a function of the electromagnetic radiation deposition information.

These and other objects, features and advantages of the present disclosure will become apparent upon reading the following detailed description of exemplary embodiments of the present disclosure, when taken in conjunction with the accompanying exemplary drawings and appended claims.

### BRIEF DESCRIPTION OF THE DRAWINGS

The foregoing and other objects of the present disclosure will be apparent upon consideration of the following detailed description, taken in conjunction with the accompanying exemplary drawings and claims showing illustrative embodiments of the invention, in which:

FIG. 1 is a diagram of an exemplary subject and RF array coil and/or antenna structure which can be viewed as a multi-port network that interacts with a plurality of sources through the ports, in accordance with certain exemplary embodiments of the present disclosure;

FIG. 2 is an illustration of an exemplary Matlab code implementing an exemplary algorithm and/or procedure for prescribing input configurations in accordance with certain exemplary embodiments of the present disclosure;

FIG. 3(a) is an illustration of an exemplary three element array coil in accordance with certain exemplary embodiments of the present disclosure;

FIG. 3(b) is a set of images of exemplary measured ΔT maps corresponding to a sequence of 11 heating steps conducted according to 11 input configurations;

FIG. 3(c) is a set of images of exemplary predicted ΔT maps in accordance with certain exemplary embodiments of the present disclosure;

FIG. 4(a) is an exemplary flow diagram showing exemplary RF hyperthermia procedure in accordance with certain exemplary embodiments of the present disclosure;

FIG. 4(b) is an exemplary flow diagram of an exemplary procedure for determining whether information from the completed calibration steps and/or subprocesses can be sufficient to allow and/or provide for the creation of an appropriate heating pattern in the subject's body, in accordance with certain exemplary embodiments of the present disclosure;

FIG. 4(c) is an exemplary flow diagram of an exemplary procedure for moving and/or calibrating elements until an exemplary optimization procedure can have sufficient and/or preferred information and/or support to produce a proper (e.g., preferred) heating pattern, in accordance with certain exemplary embodiments of the present disclosure;

FIG. 4(d) is an exemplary flow diagram showing an exemplary RF hyperthermia procedure in accordance with certain exemplary embodiments of the present disclosure;

FIG. 5 is an exemplary flow diagram showing an exemplary generation of a thermal response-based tissue contrast in accordance with certain exemplary embodiments of the present disclosure;

FIG. 6 is an illustration of an exemplary block diagram of an exemplary system in accordance with certain exemplary embodiments of the present disclosure;

FIG. 7 is an exemplary illustration of a graph showing exemplary MR thermometry and heating sequences that can calibrate and validate local temperature change model in accordance with certain exemplary embodiments of the present disclosure;

FIG. 8 are exemplary illustrations of an exemplary coil-phantom setup used for exemplary calibration and prediction testing in accordance with certain exemplary embodiments of the present disclosure;

FIG. 9 is a table of exemplary weightings applied to the array coil in an exemplary heating procedure in accordance with certain exemplary embodiments of the present disclosure;

FIG. 10 is a set of exemplary temperature difference maps used to calibrate an exemplary model in accordance with certain exemplary embodiments of the present disclosure; and

FIG. 11 are illustrations of exemplary temperature difference maps in accordance with certain exemplary embodiments of the present disclosure;

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure.

### DETAILED DESCRIPTION OF EXEMPLARY EMBODIMENTS OF THE DISCLOSURE

### Exemplary RF Energy Deposition Pattern Prediction

In order for radiation from an array coil or antenna to be focused at the tumor site without producing undesired “hot spots” elsewhere, the driving RF waveforms' amplitudes and phases can be carefully chosen. Treatment planning can be commonly accomplished with, e.g., numerical simulation-based analysis and optimization. Such planning, in addition to involving considerable computational resources and/or medical expertise, can be susceptible to a number of modeling errors that can impact the effectiveness of the treatment. For example, the complex human hemodynamic system can impact significantly the thermodynamics in a subject. The modeling of this system can be complex and difficult mathematically, and can additionally be hindered by, e.g., a lack of subject specific blood flow information. Moreover computing electromagnetic field patterns can be time challenging at higher RF frequencies, where much specific knowledge of the subject body and the array coil can be typically required for accurate field pattern prediction.

Described herein are exemplary embodiments of apparatus, systems and methods/procedures for, e.g., model-based RF energy, deposition pattern prediction using as inputs the driving phases and amplitudes, or the input configuration. For example, certain exemplary embodiments of the present disclosure can accomplish in situ calibration with a designed set of low-dose heating and MR thermometry procedures.

In magnetic resonance imaging, a magnetic resonance (“MR”) scanner commonly modulates a radio-frequency electromagnetic field when exciting spins. The modulation can be done by updating, as specified by a designed RF pulse waveform, the magnitude and phase of a Larmor-frequency sinusoidal pulse that drives a transmit channel and a transmit coil. Such exemplary update preferably occurs every Δt time increment, which can be, for example, several microseconds in practice. This exemplary modulation can be multiplied in N-channel parallel RF transmission, where N designed waveforms, sinusoidal pulses and transmit channels, as well as an N-port transmit coil, can be employed to provide enhanced support for the RF electromagnetic field modulation, giving rise to an RF field that varies both in space and in time.

A perspective to parallel transmission can be, for example, to treat the transmit channels, the transmit coil and the subject as a single system. For any Δt interval, the magnitude-phase pairs specified by multiple RF pulse waveforms, expressed with complex scalars wₚ⁽ⁿ⁾ (n=port index and p=interval index), can define the inputs to the system. There can be one input configuration per Δt interval. The B1⁺ field and the RF energy deposition can be, for example, outputs of the system. During RF transmission the B1⁺ field can interact with the spins, leading to MR signal creation and enabling MR imaging. The concomitant E field, which typically accompanies the B1 field according to the laws of electrodynamics, can induce RF energy deposition and can cause possible temperature rise in the subject.

In a particular system according to certain exemplary embodiments of the present disclosure, parallel RF transmit chains of the MR scanner can provide hardware support for performing controlled RF energy deposition for such exemplary applications as RF hyperthermia, targeted drug delivery and thermal contrast, and the MR imaging and RF measurement capability existing on the scanner facilitate guidance and delivering of thermal dose.

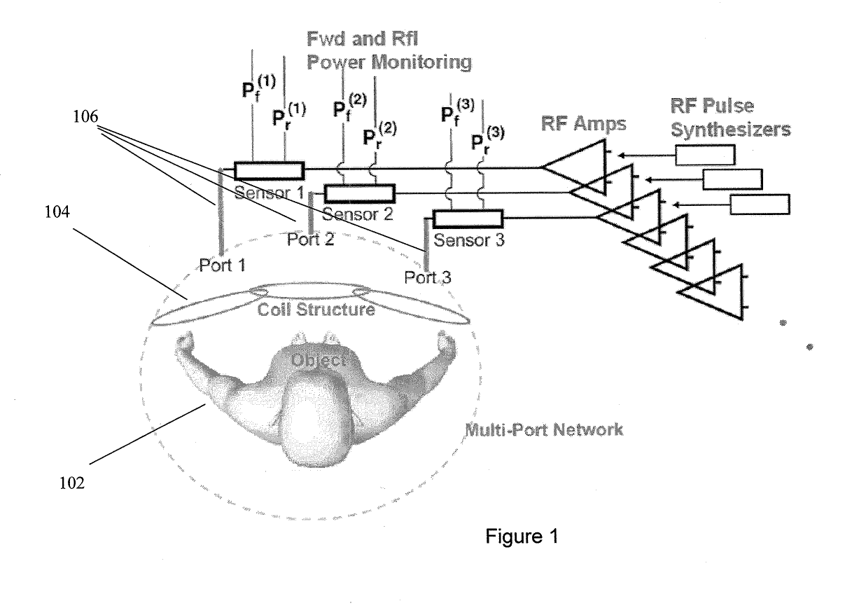

For example, in a network perspective of RF transmit where a subject 102 and RF coil and/or antenna structure/arrangement 104 can be viewed as a multi-port network that can interact with multiple sources through ports 106 (as shown, for example, in FIG. 1), an exemplary linear system relationship between the electromagnetic fields and the input configuration can indicate that the net E field can be expressed as a weighted superposition of E fields associated with the exemplary N channels employed for RF transmit. Local RF power deposition ξ_{local}, which can be caused by Joule heating and polarization damping forces, can be proportional to the square of local net E field strength: ξ_{local}=½ σ|E|², where σ=σₜᵢₛₛᵤₑ+ω∈″. Over a Δt time interval during RF transmit, local, as well as overall RF power dissipation in the N-port network, can be expressed as quadratic functions in w⁽¹⁾, w⁽²⁾, . . . and w^{(N)}. In matrix form:

local RF power deposition ξ_{local}(r)=w^{H}Λ(r)w (1)

and

global RF power deposition ξ=w^{H}Φw, (2)

where r can denote spatial location, ^{H} can denote conjugate transpose, Λ(r) and Φ can represent N-by-N positive definite Hermitian matrices, and w=[w⁽¹⁾ . . . w^{(N)}]^{T} can represent a vector collecting the magnitude-phase pairs defining the N RF pulses for the Δt time interval. In certain exemplary embodiments according to the present disclosure, Φ can be estimated through in situ experiments, procedures and/or calculations, using power sensor data collected at the ports, for example. Using the law of conservation of energy, the difference between sum of individual channel forward power and sum of individual channel reflected power Σp_{fwd}−Σp_{rfl}, the net RE power injected into the N-port network, can be equal to ξ, the overall power dissipation in the network. Given w_{q}, an input configuration for the q^{th} time interval, Σp_{fwd}−Σp_{rfl}, as computed from the sensor readings, can therefore be related to w_{q} by, for example:

Σp_{fwd,q}−Σp_{rfl,q}=w_{q}^{H}Φw_{q}=Σᵢⱼ conjugate(w_{q}⁽ⁱ⁾)w_{q}^{(j)}Φᵢⱼ, (3)

Exemplary Equation 3 can be a linear equation with Φᵢⱼ, the entries of Φ, as the unknowns, and product terms, conjugate(w_{q}⁽ⁱ⁾)w_{q}^{(j)}, as the coefficients. A performance of calibration experiments with N² or more properly prescribed input configurations played out one at a time can probe the RF loss characteristic of the multi-port network. In particular, the coefficient and power values from the experiments can facilitate exemplary Equation 3-type equations to be assembled and the entries of Φ to be determined. It is possible for this exemplary process to exclude MR imaging, and it can be completed in a fraction of a second with an automated measuring system.

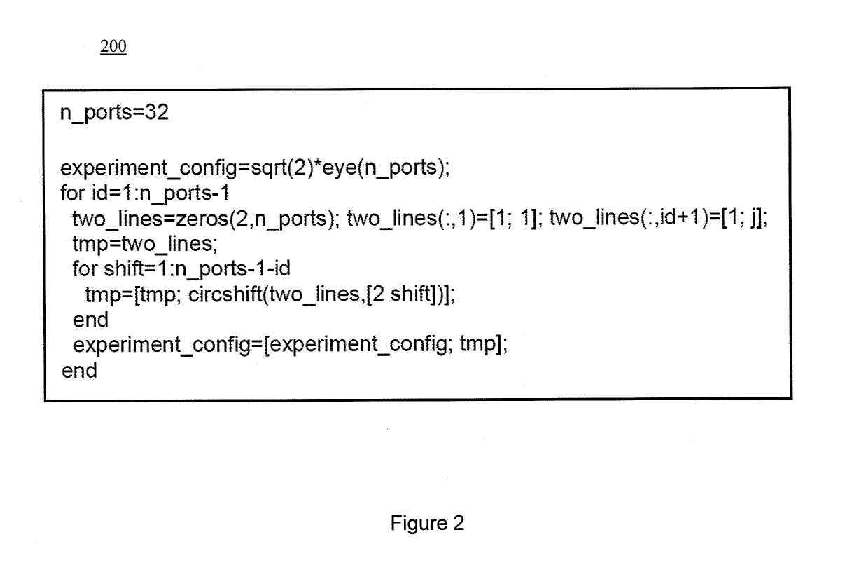

An exemplary procedure for prescribing input configurations can be used to facilitate calibration. For example, FIG. 2 shows an exemplary Matlab code 200 for implementing such exemplary procedure, prescribing a set of input configurations (e.g., result stored in experiment_config) given the number of ports (n_ports). The exemplary condition number of the resulting inverse problem can be approximately equal to n_ports, which can be a relatively low value that can be beneficial for solving the inverse problem and determining the entries of Φ. The exemplary procedure can facilitate a construction of exemplary Equation 3-type equations in such a way that a least squares solution can be robust against perturbation and/or noise, for example. When the exemplary calibration procedure is completed, the exemplary SAR prediction model can predict, for a specific subject, the exemplary SAR consequence of any (or virtually any) arbitrary input configuration and/or parallel RF transmit pulses. If and when preferred and/or desired, it can be possible for certain exemplary embodiments of the present disclosure to (e.g., also) model and/or predict individual channel forward and/or reflected power. To achieve this, exemplary Equation 3 can be modified to be in the form as follows

n^{th} channel p_{fwd,q}=w_{q}^{H}Φ_{fwd}⁽ⁿ⁾w_{q}, (4)

or

n^{th} channel p_{rfl,q}=w_{q}^{H}Φ_{rfl}⁽ⁿ⁾w_{q}, (5)

Through the calibration experiments and exemplary procedures, the n^{th} RF transmit channel's forward and reflected power transmission can be characterized. For example, the same (or similar) coefficient and power values for determining Φ can be sufficient for further determining Φ_{fwd}⁽ⁿ⁾'s and Φ_{rfl}⁽ⁿ⁾'s, and thereby characterize the individual RF transmit channel's forward and reflected power. For example, exemplary predicted values of the n^{th} channel's forward and reflected power for an arbitrary input configuration w can be, respectively, w^{H}Φ_{fwd}⁽ⁿ⁾w and w^{H}Φ_{rfl}⁽ⁿ⁾w.

It can be possible to use a similar exemplary scheme, procedure and/or approach to, e.g., establish subject-specific local SAR prediction models and/or to predict SAR distribution. In certain exemplary embodiments according to the present disclosure, it can be possible to apply accurate MR thermometry to not only map temperature, but also to provide data for determining Λ(r). For example, this can be explained using the Pennes Bio-heat equation, which can suggest that if an RF transmit experiment and/or procedure is conducted at a time scale sufficiently short in comparison to that of the heat conduction, a local temperature rise can typically be proportional to the local RF energy deposition rate: ΔT=k ξ_{local}. At a location r inside the body of a subject, it can be possible to use the exemplary ΔT(r) measurements from MR thermometry to determine kΛ(r):

ΔT(r)=kξ_{local}(r)=w_{q}^{H}kΛ(r)w_{q}=Σᵢⱼ conjugate(w_{q}⁽ⁱ⁾)w_{q}(j)(kΛᵢⱼ(r)) (6)

As one having ordinary skill in the art should appreciate in view of the exemplary embodiments of the present disclosure described herein, the same (or substantially similar) principle and/or procedure for determining Φ as explained with respect to Equation 3 can be applied in this and other exemplary embodiments as well. One difference from certain exemplary embodiments described herein can be that it can be possible to use ΔT from thermometry as sensor data in solving the linear equations for Λᵢⱼ. This exemplary method and/or procedure has been performed, for example, in simulation and phantom studies.

### Exemplary Calibration Procedure

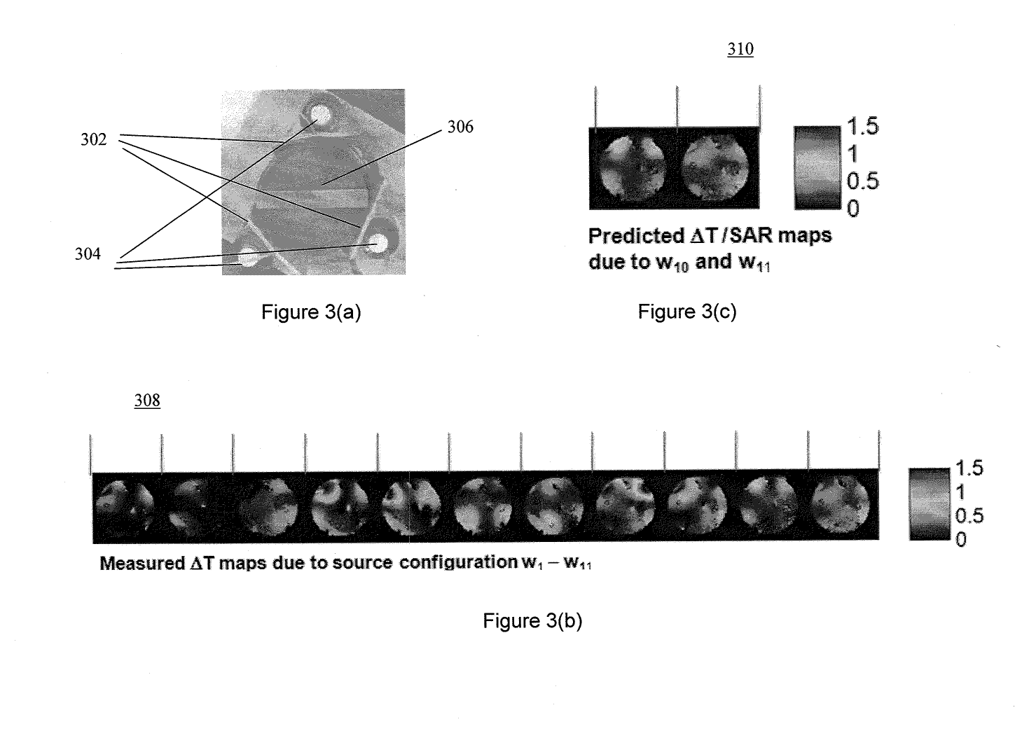

For example, FIGS. 3(a)-3(c) are illustrations that show, e.g., exemplary configuration and exemplary results according to certain exemplary embodiments of an exemplary phantom study (e.g., phantom validation example). The equation below models the exemplary temperature change in a perfusionless phantom:

$${C}_{p} ( r ) \frac{\partial T ( r )}{\partial t} + κ ( r ) {∇}^{2} T ( r ) = SAR ( r )$$

where Cₚ(r) can be the heat capacity at the r-th position, κ can be the thermal diffusivity coefficient at the r-th position, T can be the temperature at the r-th position, and t can be the time. If an RF transmit experiment and/or procedure is conducted at a time scale sufficiently short in comparison to that of the heat conduction, a local temperature rise can typically be proportional to the local RF energy deposition rate.

FIG. 3(a) shows an illustration of exemplary three element array coil 302 that can be used, for example, to perform in an interleaved manner, heating and imaging of a phantom that mimics muscle's dielectric properties. For example, three oil bottles 304 can be placed around the phantom 308 as reference, since they are typically nonconductive and likely do not heat up. FIG. 3(b) shows is an illustration of an exemplary procedure 308 of 11 heating steps/procedures conducted according to 11 input configurations (e.g., 9 for Λ(r) calibration and 2 randomly prescribed ones for assessing prediction accuracy). In each heating step/procedures, e.g., a 30% duty-cycle RF sequence was applied for 150 sec to induce modest heating (peak ΔT<1° C.). Proton resonance frequency shift-based MR thermometry (PRF) data collected before and after each heating produced a ΔT map for the step/procedure. Λ(r) can be estimated voxel by voxel using the 9 ΔT maps (the first 9 in FIG. 3(b)) from the 9 calibration steps. FIG. 3(c) is an illustration of exemplary predicted ΔT maps 310 (e.g., local SAR distribution) for the 2 randomly prescribed heating steps, which can be in agreement with the corresponding measured ΔT maps (the last 2 shown in FIG. 3(b)), demonstrating high quality prediction of temperature changes throughout the slice.

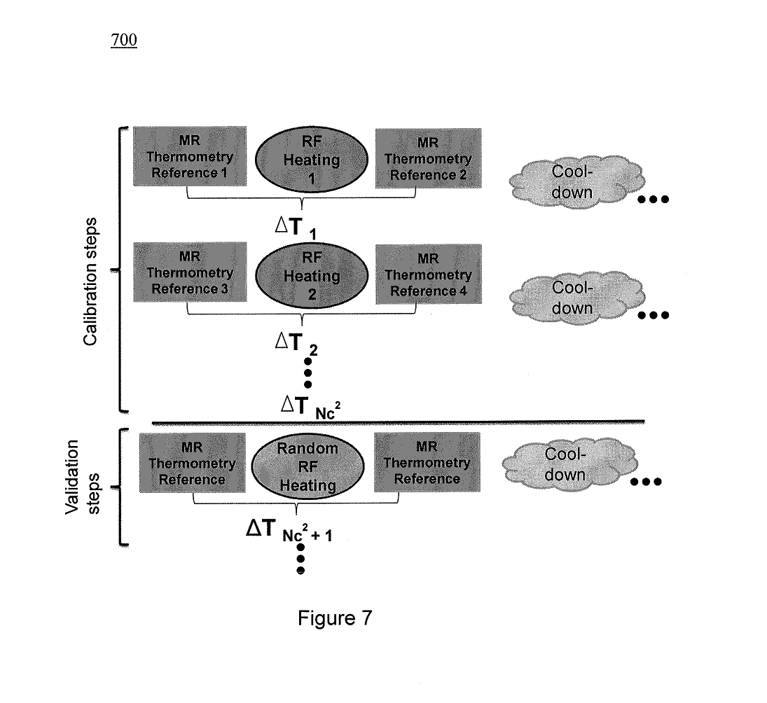

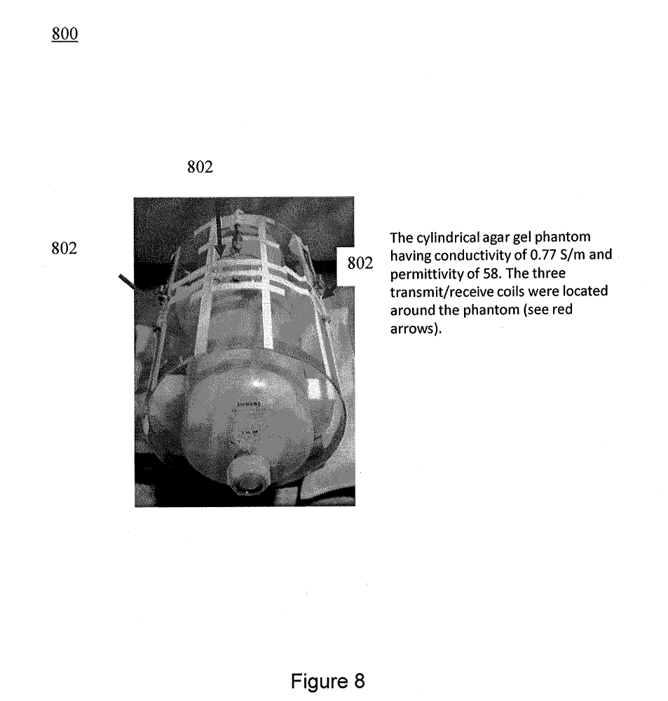

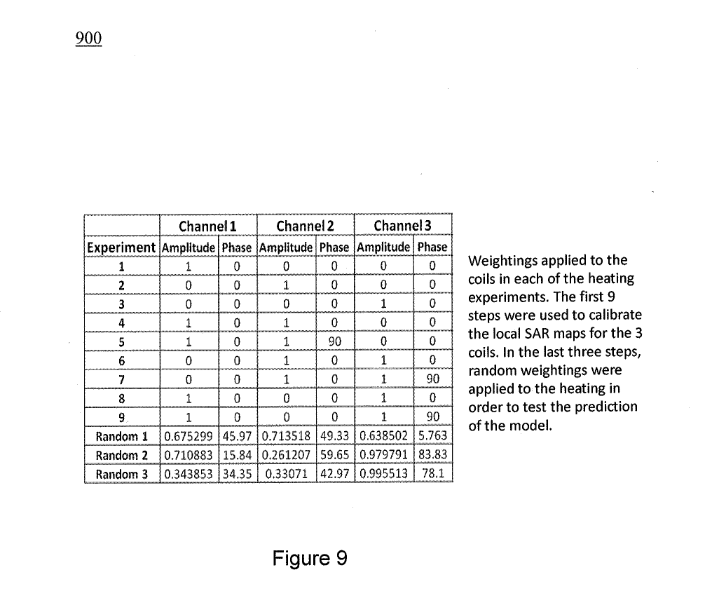

Additional exemplary experiments were performed, for example on an agar gel phantom using a three-channel transmit coil and an automated pulse sequence and post-processing. All measurements were performed, for example on a 7T scanner equipped with an 8-channel parallel transmit system. To map local SAR for the three-element transmit array, kΛ(r) can be determined using 9 calibration steps, wherein a high duty cycle RF heating pulse can be applied with various weightings for each of the steps/procedures (see, e.g., FIG. 9). Three additional steps with random transmit weightings can be performed to test the predictive capability of the exemplary model. The weightings can be defined, for example in an external file and incorporated into the sequence before runtime. To produce measurable RF heating for this gel phantom, a high-amplitude 4 ms rectangular RF pulse was played-out, for example, at 25% duty cycle for 480 seconds. Gradient pulses were not applied during this period to eliminate possible gradient-induced phase drift. For each RF heating step, a map of temperature change can be acquired using a proton resonance frequency shift (PRF) procedure. Assuming that heating happens for a relatively short period, heat diffusion can become insignificant (see, e.g., Cline H. et al. RadioFrequency Power Deposition Utilizing Thermal Imaging. Magn Reson Med 2004; 51:1129-1137) and thus ΔT(r)=k SAR(r). The temperature maps can be calculated using the following equation:

$$Δ T ( r ) = \frac{Δφ ( r )}{α \cdot TE \cdot ω} ,$$

where Δφ can be the difference in unwrapped phase between spoiled GRE images acquired before and after the RF heating period, α=0.01 ppm/° C. can be the PRF change coefficient. For each GRE acquisition, three transverse slices can be imaged, for example, with the following parameters: TR=60 ms, TE=7 ms slice thickness=8 mm, and matrix size=128×128. Between any two steps, no imaging or RF was played out for 9.6 minutes to allow the phantom and the coil electronics to cool off (e.g., FIG. 7).

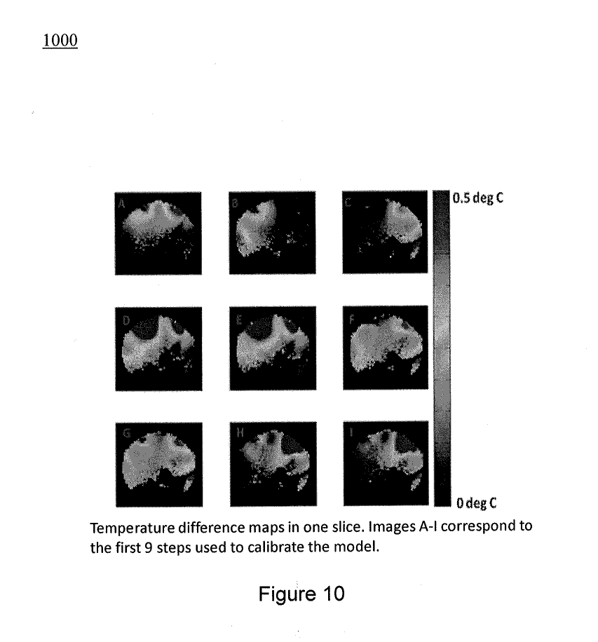

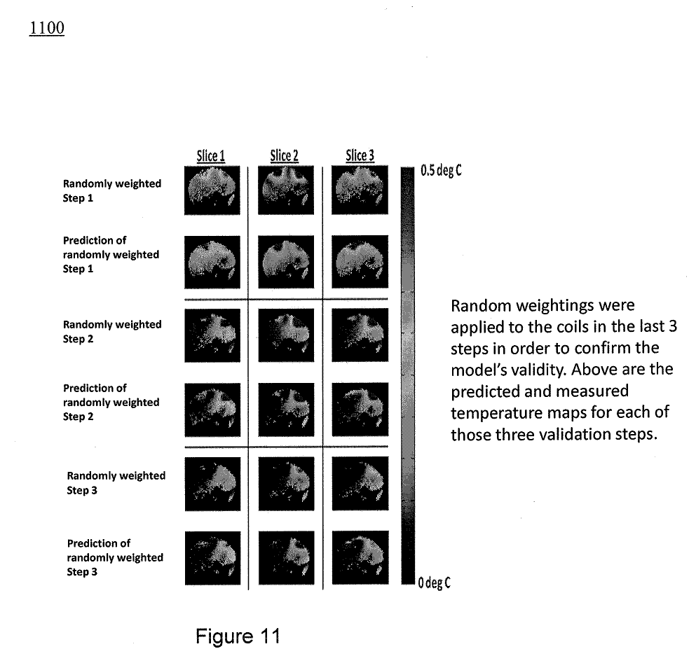

The exemplary pulse sequence streamlined GRE phase mapping and RF heating, for example to map local SAR. For the exemplary three-element transmit array coil, phase difference mapping and RF heating can be repeated 9 times with predefined transmit weightings to measure kΛ(r), plus three additional steps with random transmit RF weightings to compare the measured temperature change maps and the predicted maps calculated using the local SAR model. Results indicated excellent agreement between measurements and predictions. (See, e.g., FIGS. 10 and 11).

### Exemplary RF Hyperthermia Treatment Planning and Execution

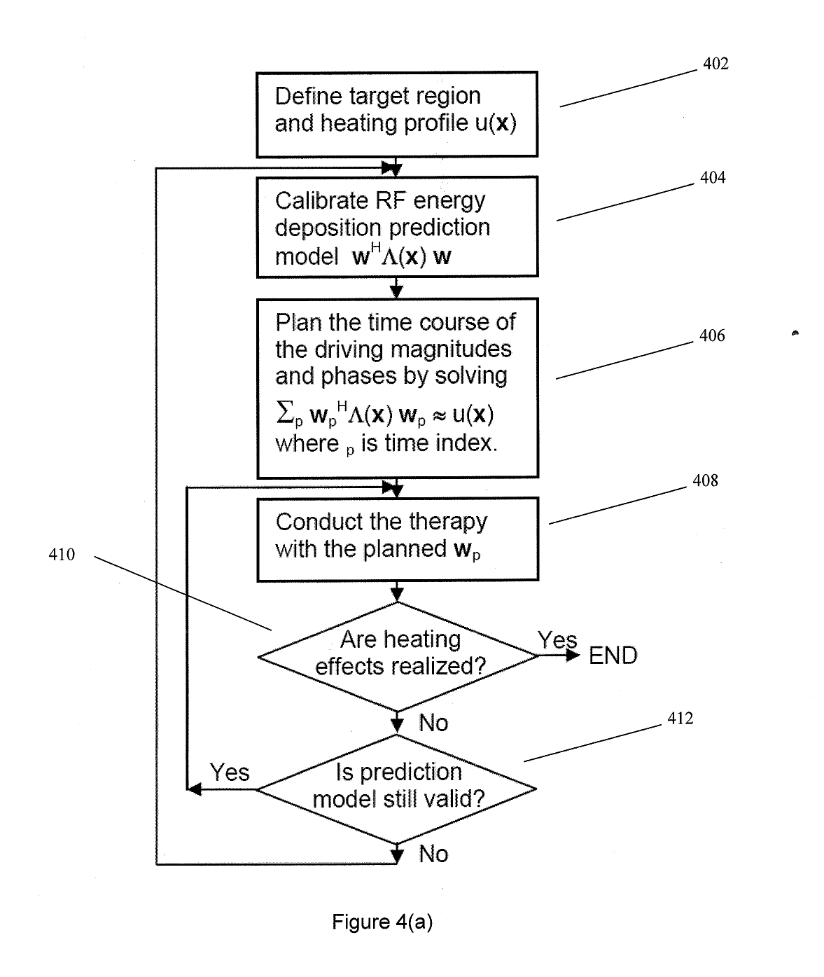

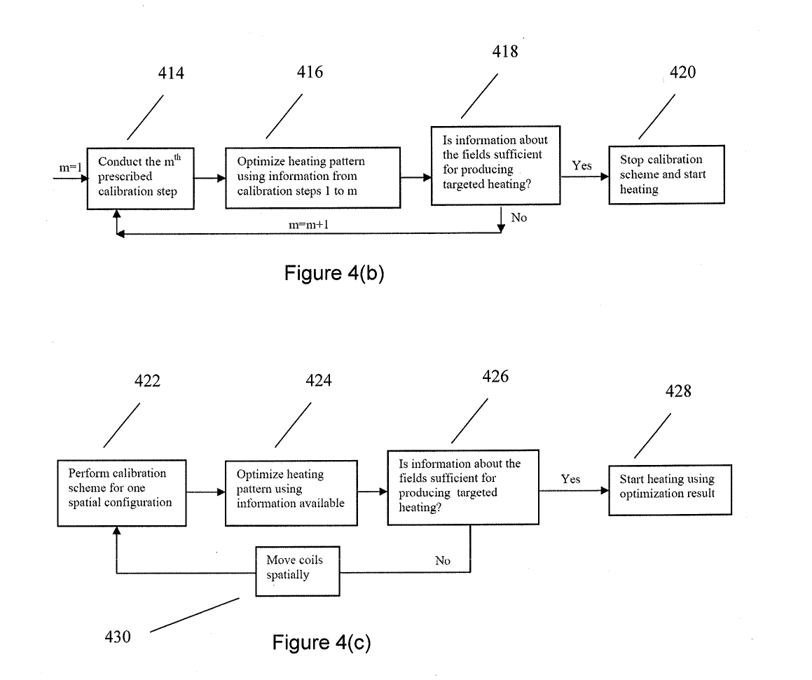

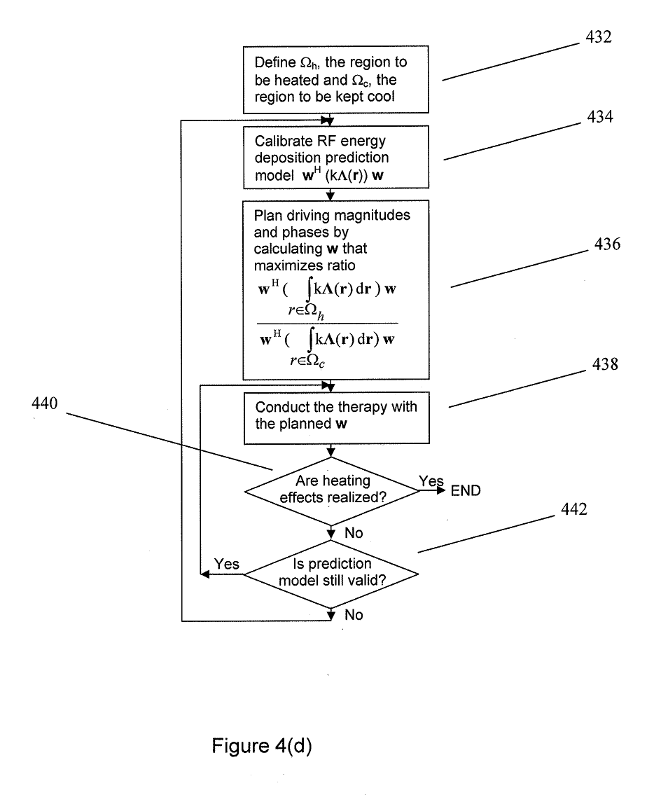

According to another exemplary embodiment in accordance with the present disclosure, it is possible to integrate an exemplary prediction-based planning procedure into an exemplary adaptive treatment process that can continuously check heating pattern and therapeutic effects, and to update the exemplary model and the planning if and when necessary and/or preferred, as illustrated in FIG. 4(a), for example.

As shown in the exemplary flow diagram of FIG. 4(a), which can be executed by a system comprising one or more processors and stored on a non-transitory computer-accessible medium according to the exemplary embodiments of the present disclosure, for example, a target region and heating profile can be defined (procedure 402). Next, an RF energy deposition prediction model can be calibrated (procedure 404). Time courses of driving magnitudes and phases can be planned (406), and the therapy can be conducted (procedure 408). Afterwards, whether the heating effects are realized (410) and whether the prediction model is still valid can be verified (procedure 412). As an alternative to targeting a specified heating pattern profile, maximizing the ratio of the RF energy deposition in a region that desires heating to the RF energy deposition in a region that desires no thermal dose, is illustrated, for example, in FIG. 4(d).

Compared to simulation-based planning and treatment, certain exemplary embodiments of the procedure according to the present disclosure can predict and/or plan energy deposition pattern (e.g., the driving force of local temperature rise), and over the course of treatment, the exemplary MRI can provide direct information for updating the prediction model and/or treatment planning periodically to account for physiological changes, as well as for assessing the therapeutic response, for example.

For exemplary N coil elements, the number of exemplary calibration steps that can be involved to model power deposition in the region of interest can be N². Therefore, as the number of elements increases, the time it can take to perform an exemplary calibration procedure can increase quadratically. According to certain exemplary embodiments of the present disclosure, it is possible to speed up calibration, for example, using a screening procedure involving: first, checking the N elements of an exemplary array and determining whether their field distributions interact with the region of interest, and second, excluding elements that do not interact with the region of interest from the subsequent calibration. It is also possible to perform the calibration iteratively. For example, after each calibration step/procedure/subprocess, it is possible to update the exemplary optimization to determine whether information from the calibration steps thus far can be sufficient to allow and/or provide for the creation of a good (e.g., optimal, preferred, etc.) heating pattern in the subject's body, as shown in a flow diagram of FIG. 4(b) which can be executed by a system comprising one or more processors and stored on a non-transitory computer-accessible medium according to the exemplary embodiments of the present disclosure.

For example, as illustrated in FIG. 4(b), an exemplary hardware processing arrangement in accordance with an exemplary procedure according to the present disclosure can conduct the m^{th} prescribed calibration step (e.g., subprocess or procedure) (414). The hardware processing arrangement can then optimize a heating pattern using information from calibration steps/procedures/subprocesses 1 to m (416). It can then be determined whether the information about the fields is sufficient for producing targeted heating (procedure 418). If yes, the hardware processing arrangement can stop the calibration and start the heating process (procedure 420). If no, then m can be incremented (e.g., m=m+1) and more elements and exemplary calibration steps can be employed for delivering a more accurate RF heating pattern.

As also described herein, yet another exemplary method/procedure in accordance with the present disclosure can be provided that can be used to facilitate a potential reduction in the number of calibration steps/subprocesses that can be used with a smaller number of coil elements (e.g., a few, 2-6, 3-4, etc.) at a time for an exemplary heating process. Over the course of the exemplary process, it is possible to alter the coil spatial configuration relative to the patient. For example, this can be realized each time by selecting a few elements from a fixed array of elements and/or translating and/or rotating the same few elements relative to the subject/patient and/or one another. In calibrating each spatial configuration, the exemplary spatial distribution of the fields can be recorded, as well as the absolute location of the individual elements. In order to move the elements from place to place, it is possible to use an MR-compatible robot that can be designed to perform accurate translations and rotations, for example. In this case the elements can be moved around and/or calibrated until an exemplary optimization procedure can have sufficient and/or preferred information and/or support to produce a proper (e.g., preferred) heating pattern, as illustrated in a flow diagram of FIG. 4(c) which can be executed by a system comprising one or more processors and stored on a non-transitory computer-accessible medium according to the exemplary embodiments of the present disclosure.

For example, as illustrated in FIG. 4(c), an exemplary hardware processing arrangement in accordance with an exemplary procedure according to the present disclosure can perform calibration for one spatial configuration (procedure 422). The hardware processing arrangement can then optimize a heating pattern using available information (procedure 424). It can then be determined whether the information about the fields is sufficient for producing targeted heating (procedure 426). If yes, the hardware processing arrangement can start the heating process using the optimized result (procedure 428). If no, then the elements can be moved spatially and the exemplary procedure repeated accordingly (procedure 430).

### Exemplary Thermal Response-Based Tissue Contrast

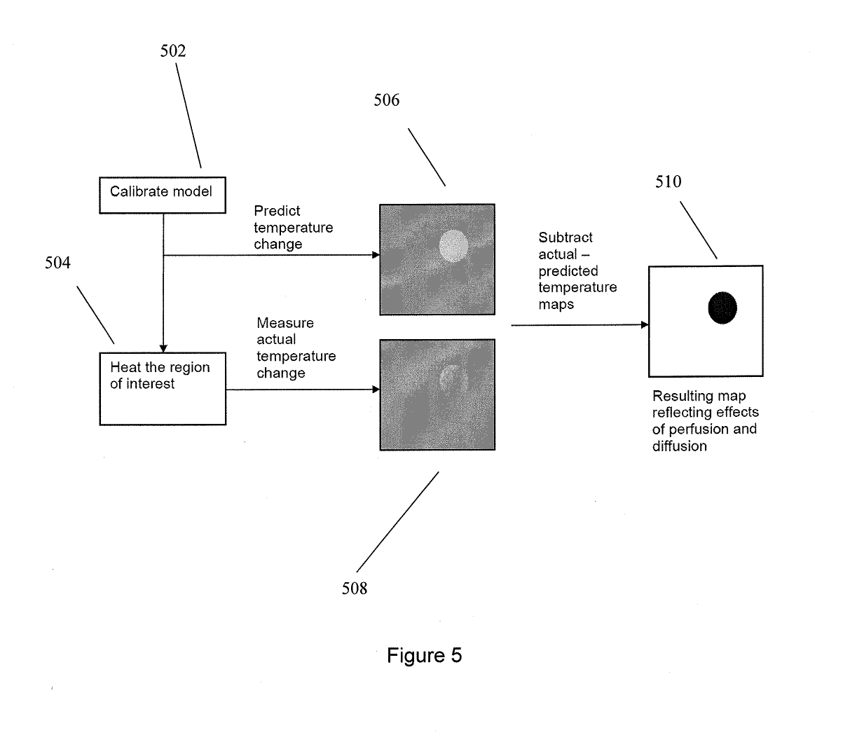

The vasodilation response to heating can vary in different types of tissue. For example, it can be expected that response to heating in cancerous tissue can be unique since cancerous tissue has generally higher IFP values and functionally does not typically support internal homeostasis. It is possible to use an exemplary local power deposition model in accordance with certain exemplary embodiments of the present disclosure to delineate the differences in the response to heating between normal tissue and cancerous tissue. FIG. 5 is an exemplary flow diagram showing exemplary generation of thermal response-based tissue contrast. For example, by subtracting the actual temperature change map (e.g., that was associated with, a slow or high-dose heating) from the predicted temperature change map (e.g., that was calculated using the exemplary prediction model from a fast and low-dose heating calibration in accordance with exemplary embodiments of the present disclosure), a measure of the differences in response to heating between different types of tissues can be obtained (procedure 510). This exemplary contrast measure can for example, encapsulate both perfusion and diffusion effects that can be induced by the exemplary heating procedure and can be reflective of the differences in the response to heating between normal tissue and cancerous tissue. As shown in FIG. 5, the model can first be calibrated (procedure 502) and the region of interest can be heated (procedure 504), prior to prediction and measurement of the temperature changes can both be obtained (procedures 506 and 508) and the contrast can be produced (procedure 510).

### Exemplary System

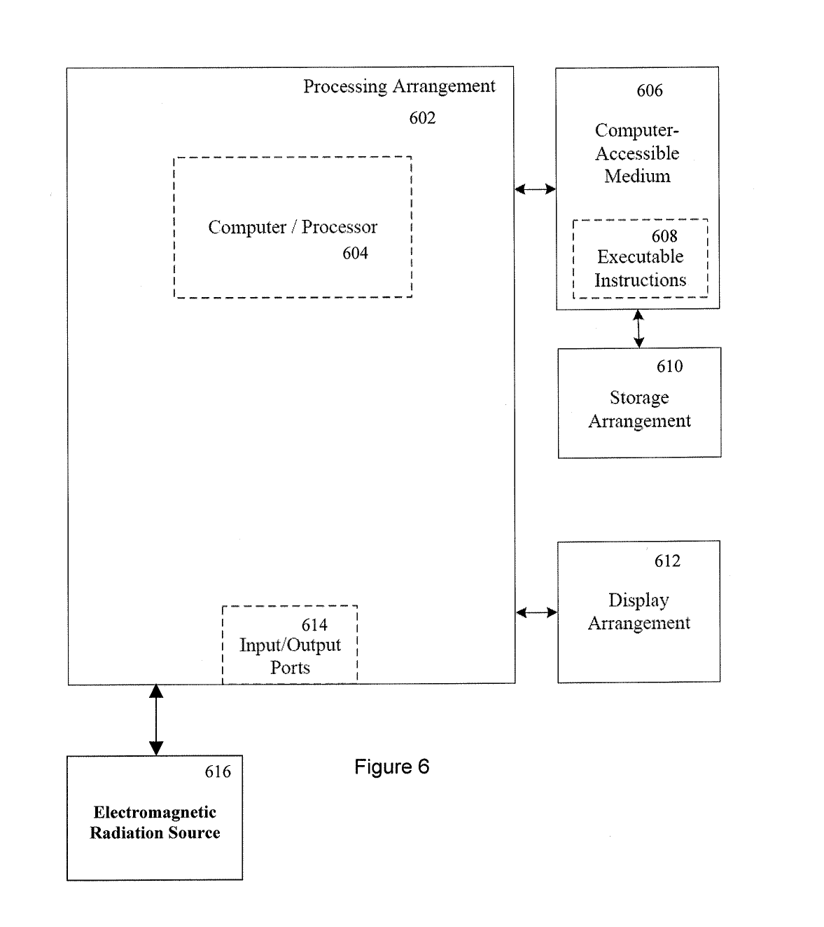

FIG. 6 shows an exemplary block diagram of an exemplary embodiment of a system according to the present disclosure. For example, exemplary procedures in accordance with the present disclosure described herein can be performed by a processing arrangement and/or a computing arrangement 602 and an RF source 616. Such processing/computing arrangement 602 can be, e.g., entirely or a part of, or include, but not limited to, a computer/processor 604 that can include, e.g., one or more microprocessors, and use instructions stored on a computer-accessible medium (e.g., RAM, ROM, hard drive, or other storage device).

As shown in FIG. 6, e.g., a computer-accessible medium 606 (e.g., as described herein above, a storage device such as a hard disk, floppy disk, memory stick, CD-ROM, RAM, ROM, etc., or a collection thereof) can be provided (e.g., in communication with the processing arrangement 602). The computer-accessible medium 606 can contain executable instructions 608 thereon. In addition or alternatively, a storage arrangement 610 can be provided separately from the computer-accessible medium 606, which can provide the instructions to the processing arrangement 602 so as to configure the processing arrangement to execute certain exemplary procedures, processes and methods, as described herein above, for example.

Further, the exemplary processing arrangement 602 can be provided with or include an input/output arrangement 614, which can include, e.g., a wired network, a wireless network, the internet, an intranet, a data collection probe, a sensor, etc. As shown in FIG. 6, the exemplary processing arrangement 602 can be in communication with an exemplary display arrangement 612, which, according to certain exemplary embodiments of the present disclosure, can be a touch-screen configured for inputting information to the processing arrangement in addition to outputting information from the processing arrangement, for example. Further, the exemplary display 612 and/or a storage arrangement 610 can be used to display and/or store data in a user-accessible format and/or user-readable format.

The foregoing merely illustrates the principles of the present disclosure. Various modifications and alterations to the described embodiments will be apparent to those having ordinary skill in art the in view of the teachings herein. It will thus be appreciated that those having ordinary skill in art will be able to devise numerous systems, arrangements, and methods which, although not explicitly shown or described herein, embody the principles of the disclosure and are thus within the spirit and scope of the disclosure. In addition, all publications and references referred to above are incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement which can be a microprocessor, mini, macro, mainframe, etc. In addition, to the extent that the prior art knowledge has not been explicitly incorporated by reference herein above, it is explicitly being incorporated herein in its entirety. All publications referenced above are incorporated herein by reference in their entireties.

1. A method for obtaining information associated with a deposition of at least one electromagnetic radiation in a subject using a plurality of transmit elements, comprising:
directing the at least one electromagnetic radiation at or to the subject using the transmit elements; and
with a processor arrangement, obtaining the information within the subject using a magnetic resonance (MR) scanning apparatus.

2. The method of claim 1, wherein the at least one electromagnetic radiation includes a radio frequency (RF) energy.

3. The method of claim 1, wherein the information is determined as a function of at least one of a constructive interference of the transmit elements or a destructive interference of the transmit elements.

4. The method of claim 1, wherein the information includes further information associated with a relationship between the transmit elements and the subject.

5. The method of claim 1, wherein the directing procedure comprises controlling at least one of a phase or a magnitude associated with a current of at least one of the transmit elements.

6. A method for generating a prediction model for at least one of an electromagnetic radiation absorption or a specific absorption rate (SAR), comprising:
directing at least one electromagnetic radiation at a subject using a plurality of transmit elements;
obtaining electromagnetic radiation deposition information associated with a deposition of the at least one electromagnetic radiation within the subject using a sensing apparatus; and
with a processor arrangement, generating the prediction model as a function of the electromagnetic radiation deposition information.

7. The method of claim 6, wherein the at least one electromagnetic radiation includes a radio frequency (RF) energy.

8. The method of claim 6, wherein the directing procedure comprises controlling at least one of a phase or a magnitude associated with a current of at least one of the transmit elements.

9. The method of claim 8, wherein the generating procedure comprises converting the electromagnetic radiation deposition information and the at least one phase or magnitude to the prediction model.

10. The method of claim 6, wherein the electromagnetic radiation deposition information comprises actual temperature data.

11. The method of claim 10, wherein the actual temperature data is obtained using at least one of a magnetic resonance thermometry source, an infrared source, or an embedded temperature sensing source.

12. The method of claim 6, further comprising at least one of (i) facilitating a delivery of a predetermined dose of the electromagnetic radiation to the subject based on the prediction model, (ii) predicting a local electromagnetic radiation power deposition as a function of at least one driving waveform and the prediction model, (iii) controlling at least one of a phase or an amplitude of waveforms of at least one of the transmit elements as a function of the prediction model so as to perform an electromagnetic radiation deposition, (iv) facilitating a delivery and release of a drug to the subject, wherein the release of the drug is based on a spatially discriminating dose of the electromagnetic radiation to be provided to the subject.

13. (canceled)

14. The method of claim 12, wherein the at least one driving waveform is configured to shape a spatial distribution of the local electromagnetic radiation power deposition.

15. The method of claim 14, wherein the spatial distribution of the local electromagnetic radiation power deposition is configured to at least one of (i) avoid a substantial concentration of electromagnetic radiation power deposition, or (ii) target a pre-defined electromagnetic radiation power deposition profile.

16-17. (canceled)

18. The method of claim 12, further comprising monitoring a progress of the electromagnetic radiation energy deposition.

19. The method of claim 12, wherein the controlling procedure facilitates the electromagnetic radiation energy deposition and provides a hyperthermia treatment.

20. The method of claim 19, wherein the hyperthermia treatment is configured to treat at least one of a tumor, arthritis or deep tissue heating.

21. The method of claim 12, further comprising:
determining further information associated with a relationship of a normal response of biologic tissue to an electromagnetic radiation to be provided to the subject and an abnormal response to the electromagnetic radiation, wherein the further information is based on a pathology of an abnormal tissue; and
determining, using the further information, a contrast configured to be used for diagnostic purposes.

22. (canceled)

23. The method of claim 18, wherein the monitoring procedure includes implementing at least one of magnetic resonance imaging or thermometry.

24. A method for facilitating magnetic resonance imaging, comprising:
determining a local RF power deposition model associated with a subject;
inducing heat inside tissue in a region of interest of the subject;
using a computing arrangement, subtracting a measured heat induced from a predicted heat associated with a plurality of voxels; and
determining a map associated with at least one of a perfusion or a diffusion of the region of interest as a function of the subtraction.

25. The method of claim 24, further comprising obtaining the measured heat induced at least one of (i) from a magnetic resonance thermometry associated with the subject, or (ii) using at least one of a diffusion weighted imaging procedure associated with the subject or a proton resonance frequency shift associated with the subject.

26-27. (canceled)

28. A computer-accessible medium having instructions thereon for generating a prediction model for at least one of an electromagnetic radiation absorption or a specific absorption rate (SAR), wherein, when a hardware processing arrangement executes the instructions, the hardware arrangement is configured to:
direct at least one electromagnetic radiation at a subject using a plurality of transmit elements;
obtain electromagnetic radiation deposition information associated with a deposition of the at least one electromagnetic radiation within the subject using a sensing apparatus; and
generate the prediction model as a function of the electromagnetic radiation deposition information.

29. A system for generating a prediction model for at least one of an electromagnetic radiation absorption or a specific absorption rate (SAR), comprising:
a computer-accessible medium having executable instructions thereon, wherein when at least one hardware processing arrangement executes the instructions, the at least one hardware processing arrangement is configured to:
direct at least one electromagnetic radiation at a subject using a plurality of transmit elements;
obtain electromagnetic radiation deposition information associated with a deposition of the at least one electromagnetic radiation within the subject using a sensing apparatus; and
generate the prediction model as a function of the electromagnetic radiation deposition information.
